(19) United States
(12) Patent Application Publication
(10) Pub. No.: US 2007/0073237 A1
(43) Pub. Date: Mar. 29, 2007

(54) Cannula/catheter introducer

(76) Inventor: Aaron Rodd, Queensland (AU)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/565,734

(22) PCT Filed: Jul. 30, 2004
(86) PCT No.: PCT/AU04/01012

§ 371 Date: Jan. 24, 2006

(30) Foreign Application Priority Data
Jul. 31, 2003 (AU) 2003903990

(51) Int. Cl. A61M 5/178

(52) U.S. Cl. 604/164.01

(57) ABSTRACT

A device is disclosed that allows a puncture needle to be inserted into a catheter. Once the puncture needle has performed its function, it can shoot back into the device such that there is a reduce likelihood of needlestick injury. Vacuum is used to assist in the shoot back function.

### FIELD OF THE INVENTION

The present invention relates to cannula or catheter introducer devices and has particular relevance to such devices with retractable needles.

### BACKGROUND ART

Catheter insertion devices are well known in the art. When a catheter is inserted into a patient for the intravenous delivery of a fluid, a disposable needle passing through the catheter cannula is utilized to make a puncture to permit entry of the tip of the cannula into the patient. The needle is then withdrawn leaving the catheter in place either for a direct hook-up to a bottle of fluid to be delivered, or to be capped for later use. The needle which is now contaminated with blood or other body fluid must be disposed of without creating a risk of needle sticks to medical personnel which are engaged in the operation of inserting a catheter. A danger to clean up and medical waste disposal personnel continues if the used needles are not rendered harmless in some way. Caps or covers that can be mounted over the needle are not a satisfactory solution because they must be put in place and can become loose and expose the used needle.

A discussion of the problems associated with various approaches and uses of cannula insertion products is found in Kulli, U.S. Pat. No. 4,747,831.

Retractable needles have been recognized as the best solution to these problems.

A number of these approaches are disclosed in U.S. Pat. No. 4,747,831 mentioned above which includes an external latch mechanism which is pushed to release a spring loaded needle which is withdrawn into the chamber of the device. Some embodiments have a sliding block and retractable fingers which depress springloaded ears to allow retraction of the needle holder, racheting devices which unlatch the needle holder by rotation of parts and even frangible parts which are broken when a plunger is pushed forward.

Dysarz, U.S. Pat. No. 5,129,884, is another example of an external latch which may be disengaged to allow a needle holder to withdraw a needle into the main body of the device. Walter, U.S. Pat. No. 4,828,548, discloses a holder having a piston which may be operated on by vacuum to withdraw a needle. Erskine, U.S. Pat. No. 5,501,675, is a more recent version of a latch-operated device wherein a needle holding part may be released with an external latch.

The devices of the prior art have complicated parts which are difficult to manufacture and especially difficult to assemble in a high speed manufacturing operation. Many of them are prone to premature firing during handling and with some, it is difficult to know when the needle is safe. Disengagement of the catheter assembly from the retraction device is not fully under the timing and control of the medical operator because retraction results in immediate separation of the device form the cannula assembly.

It is an object of the present invention to provide a simplified cannula/catheter introducer which is safe to use and can be produced at a modest cost and which uses a vacuum to retract a contaminated needle to reduce needle stick injury.

Further objects and advantages of the present invention will become apparent from the ensuing description which is given by way of example only.

### DISCLOSURE OF INVENTION

In one form, the invention resides in a cannula/catheter introducer comprising an outer tubular member which has a proximal end and a distal end,
an inner tubular member adapted for sliding movement within the outer tubular member, the inner tubular member having an open proximal end, and a closed distal end,
an end member which closes the open proximal end of the inner tubular member,
a sealing means on the end member to sealingly engage with the inner tubular member,
release means on the end member and which is movable between a first position where the release means locks the end member to the inner tubular member, and a second position where the release means is unlocked from the inner tubular member and allows the end member to retract through the inner tubular member, the inner tubular member being under vacuum, the vacuum functioning to retract the end member from the proximal end of the inner tubular member towards the distal end of the inner tubular member, the release means being movable from the first position to the second position upon movement of the inner tubular member towards the proximal end of the outer tubular member,
a needle holder which is attached to the proximal end of the outer tubular member, release means on the needle holder and which is moveable between a first position where the release means locks the needle holder to the outer tubular member, and a second position where the release means is unlocked from the inner tubular member,
wherein, upon movement of the inner tubular member towards the proximal end of the outer tubular member, the release means on the end member unlocks from the inner tubular member, and locks against the release means on the needle holder, and causes the release means on the needle holder to move to the unlocked position, after which the end member and the needle holder is retracted into the inner tubular member by virtue of the vacuum in the inner tubular member.

In another form, the invention resides in a cannula/catheter introducer comprising an outer tubular member which has a proximal end and a distal end,
an inner tubular member adapted for sliding movement within the outer tubular member, the inner tubular member having an open proximal end and a closed distal end,
an end member which closes the open proximal end of the inner tubular member,
a sealing means on the end member to sealingly engage with the inner tubular member,
release means on the end member and which is movable between a first position where the release means locks the end member to the inner tubular member, and a second position where the release means is unlocked from the inner tubular member and allows the end member to retract through the inner tubular member, the inner tubular member being under vacuum, the vacuum functioning to retract the end member from the proximal end of the inner tubular member towards the distal end of the inner tubular member, the release means being movable from the first position to the second position upon movement of the inner tubular member towards the proximal end of the outer tubular member,
a needle holder which is attached to the proximal end of the outer tubular member, release means on the needle holder and which is moveable between a first position where the release means locks the needle holder to the outer tubular member, and a second position where the release means is unlocked from the inner tubular member,
the inner tubular member being movable between a retracted position and an extended position, and when in the retracted position, the tubular member is spaced from the needle holder by a short distance, and when in the extended position, the tubular member triggers retraction of the needle holder, the inner tubular member being held captive within the outer tubular member in the retracted position,
wherein upon movement of the inner tubular member towards the proximal end of the outer tubular member, the release means on the end member unlocks from the inner tubular member, and locks against the release means on the needle holder and causes the release means on the needle holder to move to the unlocked position, after which the end member and the needle holder is retracted into the inner tubular member by virtue of the vacuum in the inner tubular member.

In this form of the invention, because the device functions to insert a puncture needle into a catheter or cannula, the device does not function as a syringe. Therefore, the inner tubular member in the initial position is substantially entirely within the outer tubular member and is only a short distance away from the needle holder. For instance, it is envisaged that the inner tubular member can be spaced between 1-20 millimetres from the needle holder, and preferably between 3-10 millimetres. As it is not desirable for the inner tubular member to be retracted too far in the outer tubular member (as the device does not function as a syringe), some form of locking arrangement can be provided to ensure that the inner tubular member can move only by a short distance between the retracted position and the forward (triggering) position.

The introducer can be of any suitable shape and size which will depend, inter alia, on the size of the cannula or catheter. It is envisaged that the introducer will be made of suitable material such as plastic although some components (e.g. the sealing means) may be made of rubber or composite materials. However, no particular limitation is to be placed on the invention merely by the material from which the device is manufactured. The size of the introducer can vary, and it is envisaged that the length will be between 50-300 millimetres and typically between 100-200 millimetres. Again, no particular limitation should be placed on the invention merely by the size of the device. The introducer may have a diameter of between 5-50 millimetres and typically between 10-20 millimetres although no particular limitation should be placed on the invention merely by the diameter of the introducer.

The outer tubular member may comprise finger grippable portions to assist in a one-handed operation in depressing the inner tubular member within the outer tubular member. The finger grippable portions may comprise extending members adjacent the proximal end of the outer tubular member and dimensioned to enable fingers to grip these portions.

The proximal end of the outer tubular portion may be restricted in diameter with respect to the remainder of the outer tubular portion. Suitably, the proximal end contains a number of stepped portions to progressively reduce the diameter of the proximal end while still providing internal land portions.

The end of the proximal end of the outer tubular portion may comprise a nozzle adapted for insertion into the rear of a catheter.

Gripping means or locking means may be provided on an inner face at the proximal end of the outer tubular member to assist in gripping or locking the needle holder. The gripping means or locking means may comprise a recess such as an annular groove.

The distal end of the outer tubular member may be provided with a locking means to restrict movement of the inner tubular member relative to the outer tubular member. Suitably, the locking means comprises an enlargement on the outer tubular member and adjacent the distal end, the enlargement defining a small travelling passageway which defines the limits of movement of the inner tubular member relative to the outer tubular member. The passageway may have a length of between 3-20 millimetres and typically between 3-10 millimetres. The passageway may contain engagement means to “lightly” engage with the inner tubular member. The engagement means may comprise a pair of spaced apart small ribs, which may be annular in configuration. The function of the engagement means may be to lightly hold the inner tubular member in the retracted position such that the inner tubular member does not inadvertently move to the extended position thereby triggering the shoot-back mechanism prematurely. However, the engagement means should be such that a person can press on the back of the inner tubular member to overcome the engagement means when desired.

The inner tubular member, in use, will have reduced pressure, or be under vacuum. The inner tubular member will typically have a closed distal end and an open proximal end, with the open proximal end being closed by the end member. Typically, the inner tubular member will comprise a hollow tube with an open front end. The distal end of the inner tubular member will typically be provided with locking means to lock against the distal end of the outer tubular member in such a manner that the degree of sliding movement of the inner tubular member within the outer tubular member is predetermined. The locking means may comprise a projection which may be an annular rib that sits within the passageway that may be provided on the distal end of the outer tubular member.

The end member closes the otherwise open proximal end of the inner tubular member. The end member may comprise a rear portion and a front portion. The rear portion may be substantially closed and may be provided with the sealing means to enable the end member to be sealingly engaged to the inner wall of the inner tubular member in a sliding but sealing manner. The sealing means may comprise a sealing ring that is fitted about the rear portion and which comprises at least one, and preferably a plurality of, spaced-apart ring members that sealingly engage against the inside wall of the inner tubular member.

The front portion may contain or comprise the release means. The release means may be formed integrally with the rear portion. The release means may comprise at least one finger member. The finger member may be resiliently formed to move between a natural first position and a deformed second position. Suitably, the finger member deforms inwardly between the first position and the second position. Suitably, a plurality of finger members is provided and these may be spaced equally about the end member. The or each finger member may extend forwardly from the end member and towards the needle holder.

The end member may have a length of between 5-20 millimetres although this can vary to suit.

A “flash” chamber may be provided in the end member to enable the first flash of blood or body fluid to be viewed as the puncture needle penetrates into a patient's vein, etc.

The or each finger member in the natural first position may lock against the inner tubular member and preferably lock against the open proximal wall of the inner tubular member. It is preferred that the or each finger member is profiled to facilitate this type of locking. Therefore, it is preferred that the or each finger member contains a step, or a shoulder, or have an arrowhead-type configuration to enable the or each finger member to abut against or lock against the open proximal wall of the inner tubular member.

The needle holder is attached to the proximal end of the outer tubular member and is preferably positioned in the stepped or narrowing position of the outer tubular member. The needle holder may be made of any suitable material such as plastic, rubber, composite materials and the like. The needle holder will typically comprise a central body portion. The central body portion may contain a small passageway through which a puncture needle can be fitted to fit the puncture needle to the needle holder. The release means may be formed integrally with the remainder of the needle holder. The release means may comprise at least one finger member. The at least one finger member may be movable or deformable between a natural position which is the first position and a deformed position which is the second unlocking position. The at least one finger member may be cantilevered from the central body portion and may extend towards the inner tubular member. Suitably, a plurality of finger members is provided and these may be spaced equally about the central body portion. Each finger member may be provided with a terminal configuration which may comprise a shoulder, a step portion, a hook portion, or an arrowhead portion, the function of which is to facilitate engagement with the finger member on the end member as will be described in greater detail below. Also, each finger member may comprise an intermediate projection such as an annular ring which engages into the recess on the outer tubular member to temporarily lock the needle holder to the outer tubular member.

In another form, the invention resides in a cannula/catheter inserter, the inserter comprising
an outer tubular member which has a proximal end and a distal end,
an inner tubular member adapted for sliding movement within the outer tubular member, the inner tubular member having an open proximal end, and a closed distal end,
a needle holding piston assembly which closes the open proximal end of the inner tubular member,
a sealing means on the needle holding piston assembly to sealingly engage with the inner tubular member,
release means on the needle holding piston assembly and which is movable between a first position where the release means locks the needle holding piston assembly to the inner tubular member and a second position where the release means is unlocked from the inner tubular member and allows the needle holding piston assembly to retract through the inner tubular member, the inner tubular member being under vacuum, the vacuum functioning to retract the needle holding piston assembly from the proximal end of the inner tubular member towards the distal end of the inner tubular member, the release means being movable from the first position to the second position upon movement of the inner tubular member towards the proximal end of the outer tubular member,
wherein upon movement of the inner tubular member towards the proximal end of the outer tubular member, the release means on the needle holding piston assembly unlocks from the inner tubular member, after which the needle holding piston assembly is retracted into the inner tubular member by virtue of the vacuum in the inner tubular member.

It is preferred that the inner tubular member is movable between a retracted position and an extended position and when in the retracted position, the tubular member is spaced from the needle holding piston assembly by a short distance and when in the extended position, the tubular member triggers retraction of the needle holding piston assembly, the inner tubular member being held captive within the outer tubular member such that the inner tubular member can only move by the short distance.

The short distance may be as described above and will of course depend on the size of the device. However, it is the intention that the inner tubular member can move forward by only a short distance to trigger the shoot back mechanism. Typically, this distance will be between 1-20 millimetres and preferably between 3-10 millimetres.

Suitably, the inner tubular member and the outer tubular member are substantially as described above with reference to the first embodiment of the invention.

The needle holding piston assembly may have a length of between 5-30 millimetres although no particular limitation should be placed on the invention merely by this length. The piston assembly may comprise a main body which has one closed end and one open end and is substantially hollow. The closed end may be provided with an enlargement onto which the sealing member can be positioned. The sealing member may comprise any suitable type of sealing member. The open end may contain a needle mounting block. The needle mounting block typically contains a small passageway through which the puncture needle can pass to fit the puncture needle to the needle mounting block. A small flash chamber may be provided in the main body. The needle mounting block will typically contain the release means. The release means may comprise at least one finger member. Suitably, a plurality of finger members is provided. Each finger member may contain a terminal configuration, which may comprise a step, a shoulder, a hook, or an arrowhead type configuration to facilitate locking of the finger member and therefore locking of the needle holding piston to the proximal end of the inner tubular member.

In another form of the invention, there is provided a cannula/catheter introducer comprising:
(a) a cannula and catheter connector assembly,
(b) a retractable needle assembly for assembly with the cannula and catheter assembly, and
(c) a valving member connected with the cannula and catheter assembly for controlling in ingress and egress of fluids from a patient.

The cannula and catheter connector assembly may be provided with an inlet tube for releasable assembly with an outlet tube of a retractable needle.

The valving member will typically be manually adjustable from a first sealing position to a second flow position.

The valving member may comprise a plunger type valve.

Alternatively, the valving member may comprise a turn type valve.

The retractable needle may be of the type described above, and therefore may comprise an outer and inner casing, an outlet from the casing, an internal piston within the inner casing mounting a needle and a sealed compartment between the piston and a closed end of the inner casing.

The sealed compartment contains partial pressure.

The outer casing is typically provided with wings for gripping.

The piston may include a transparent vial section which enables a user to establish that the needle has been correctly inserted.

The vial may comprise a minute venting aperture.

With the present invention, the assembled catheter introducer is handled as a unit. When the operator verifies that the needle is properly inserted, one hand can hold the connector that remains attached to the patient while the other hand separates the retraction body which upon separation is automatically retracted. A portion of the hand rests against the cannula to prevent blood flow until the other hand inserts the conventional tube into the catheter connector to start the infusion of fluid. Immediately after separation, the retraction body safely contains the retracted needle and the removed part is safely discarded.

### BRIEF DESCRIPTION OF THE DRAWINGS

Embodiment of the invention will be described with reference to the following illustrations.

FIG. 1 illustrates a section view of an introducer fitted to a catheter and according to a second embodiment of the invention, and in the “use” position.

FIG. 1a illustrates a close-up view of the proximal part of the introducer and particularly illustrating the needle holding piston.

FIG. 1b illustrates a close-up view of the distal part of the introducer and particularly illustrates the mechanism that limits travel of the inner tubular member in the outer tubular member.

FIGS. 2 and 2a illustrate initiation of the “shoot back” mechanism where the inner tubular member is being pushed forwardly in the outer tubular member to deform the needle mounting block.

FIGS. 3 and 3a illustrate the needle in the shoot back position.

FIG. 4 illustrates the introducer removed from the catheter with the puncture needle safely within the confines of the inner tubular member.

FIGS. 5 and 6 illustrate section views of the catheter.

FIG. 7 illustrates a perspective view of the introducer connected to the catheter.

FIG. 8 illustrates a section view of a first embodiment of the invention with the introducer attached to a catheter and in the “use” position.

FIG. 8a illustrates a close-up of the proximal end of the introducer.

FIG. 8b illustrates a close-up of the distal end of the introducer.

FIGS. 9 and 9a illustrate the introducer being moved to trigger the shoot back mechanism.

FIGS. 9b and 10 illustrate the shoot back position where the puncture needle is safely within the confines of the inner tubular member.

FIG. 11 illustrates the introducer removed from the catheter.

FIGS. 12-15 illustrate the valve on the catheter.

FIG. 16 illustrates a perspective view of the second embodiment of the introducer attached to the catheter.

### DETAILED DESCRIPTION OF THE EMBODIMENTS

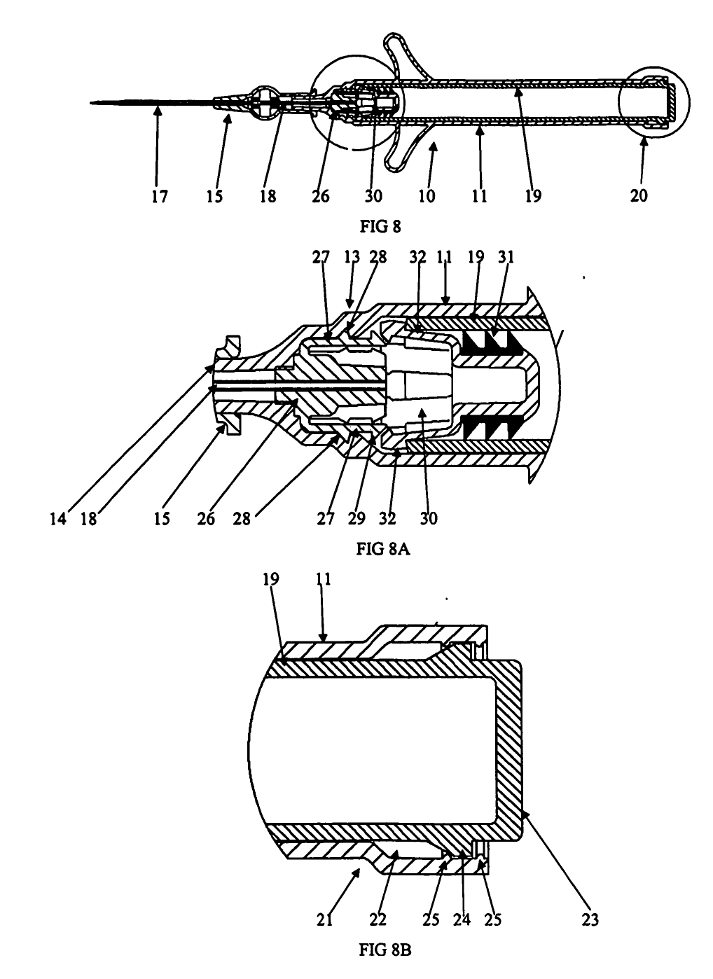

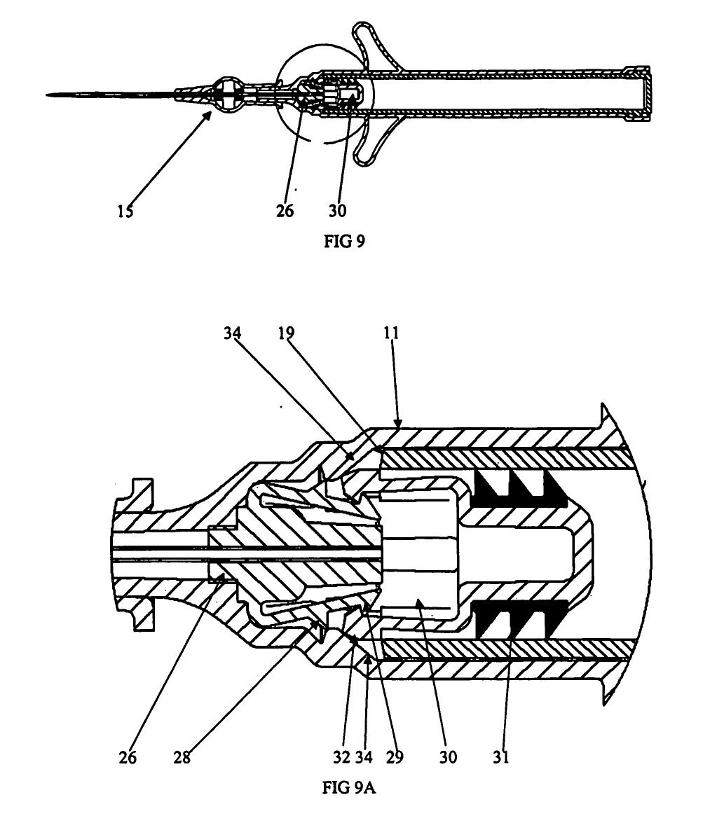

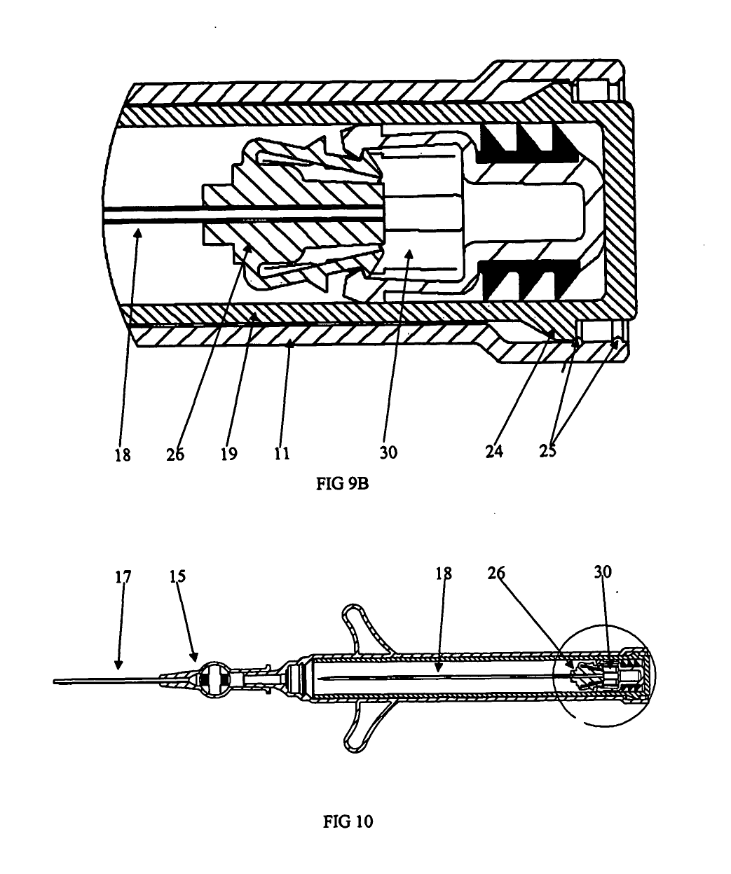

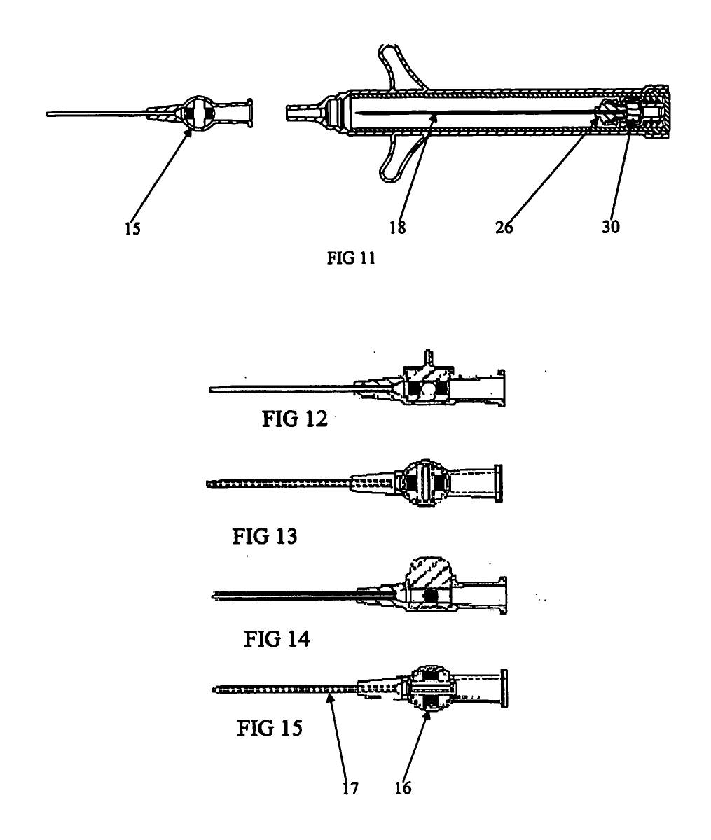

Referring to FIGS. 8-16, there is illustrated a first embodiment of the invention. Briefly, FIGS. 8-9a illustrate a cannula/catheter introducer in the “use” position, while FIGS. 9a-11 illustrate the same introducer in the “shoot back” position. FIGS. 12-15 illustrate the valve which forms part of the catheter assembly and FIG. 16 is a perspective view of the device attached to the catheter.

Referring initially to FIG. 16, there is illustrated a catheter introducer 10 which comprises an outer tube 11. Outer tube 11 contains a pair of finger gripping wings 12. The front of outer tube 11 contains a number of step portions 13 to ultimately finish in an inlet 14.

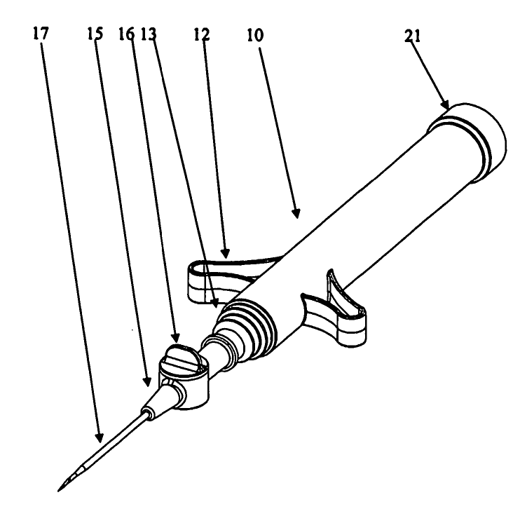

A catheter assembly 15 can be attached to inlet 14 and assembly 15 includes a valve 16 to enable the flow from the catheter needle 17 to be regulated. FIGS. 12-15 illustrate particularly valve 16. FIG. 12 is a section view showing the valve in the “off” position and FIG. 13 is a plan view showing the valve in the “off” position. FIGS. 14-15 illustrate similar views with the valve in the “on” position. The valve can be moved manually between the “on” position and the “off” position.

Referring now in greater detail to FIGS. 8, 8a and 8b, there is illustrated details of the device 10. Briefly, the device functions to enable a contaminated needle 18 to be “sucked” back into the main body of the device after use thereby preventing needle stick injury. Briefly, this is achieved by pushing the inner tube 19 forwardly relative to outer tube 11 by distance of only a few millimetres but this movement is sufficient to suck back a contaminated needle. This will now be described in greater detail.

Referring to FIGS. 8, 8a and 8b, there is illustrated the device 10 attached to a catheter assembly 15. Device 10 has an outer tube 11 and an inner tube 19 with the inner tube 19 able to slide within outer tube 11 by a short distance (typically a few millimetres). Outer tube 11 is cylindrical and can have a length of between 10-20 centimetres and a diameter of between 5-20 millimetres although no particular limitation should be placed on the invention merely by these dimensions. Outer tube 11 will typically be made of suitable plastic material. Outer tube 11 has a rear (distal) open end 20 to enable the inner tube 19 to be manipulated. The open end 20 contains an enlarged collar 21 which is best illustrated in FIG. 8b and which defines a small travelling passageway 22 of a few millimetres.

Inner tube 19 is under vacuum and has a closed rear (distal) end 23. This distal end is formed with an annular rib 24 which is designed to move along passageway 22. The annular rib 24 enables inner tube 19 to be moved forwardly (or be pressed into outer tube 11) which will cause the annular rib 24 to move from the rear position illustrated in FIG. 8b to a forward position (not illustrated) but where rib 24 has been moved to adjacent the forward end of passageway 22.

Initially, device 10 is in the position illustrated in FIGS. 8, 8a and 8b. In this position, annular rib 24 is located within a pair of spaced apart internal beads 25 (see FIG. 8b) to provide a positive location of the inner tube 19 within the outer tube 11, but which still enables a person to push the inner tube out of engagement with the beads 25 when required.

Referring to FIG. 8a, the forward part of outer tube 11 as mentioned above has a number of step portions 13 which terminate in an inlet 14. Inlet 14 is designed to attach to the rear of the catheter assembly 15 as is illustrated in FIG. 8.

The puncture needle 18 (see FIG. 8a) is secured to a needle holder 26 of special design and which forms part of the shoot back mechanism which enables needle 18 to be shot back into the rear of inner tube 19 (see FIG. 10 and FIG. 11). Needle holder 26 is initially attached to outer tube 11 but can be detached therefrom as will be described in greater detail below. Needle holder 26 contains a plurality of resilient fingers 27. Each finger 27 contains an outwardly extending rib 28 that extends into a corresponding recess on outer tube 11. This engagement locks needle holder 26 to outer tube 19 and inside the stepped area 13. The advantage of having a needle holder 26 initially locked to outer tube 11 is that there is very little chance that any rearward force on needle 18 will inadvertently release the needle holder prematurely. Also, the needle will not move forwardly during the triggering operation.

The resilient fingers 27 terminate in an arrowhead type configuration 29 and initially this part of the resilient fingers merely extends into the inside of outer tube 11 but is not attached to anything.

The other part of the shoot back mechanism comprises an end member 30. End member 30 is initially fitted to the open front (proximal) end of inner tube 19 and is best illustrated in FIG. 8a. End member 30 comprises a seal 31 which seals against the inside of inner tube 19 to maintain vacuum inside the inner tube. End member 30 is however prevented against being sucked back into inner tube 19 by a plurality of resilient fingers 32. Fingers 32 are biased to lock against the end of inner tube 19 (see FIG. 8a) and this arrangement prevents end member 30 from being sucked back into innertube 19.

The resilient fingers on end member 30 also have an arrowhead type configuration, but the arrangement is such that these resilient fingers are able to lock against the arrowhead configuration 29 on resilient fingers 27 which form part of needle holder 26. However, this does not occur until such time as inner tube 19 is pushed forwardly.

In use, a catheter assembly 15 can be fitted to the end of device 10 as illustrated in FIG. 8 and FIG. 16. The catheter can then be inserted into a person's vein. The puncture needle 18 becomes contaminated during this procedure. Once the catheter assembly 15 is in place, the conventional technique of removing the puncture needle results in a needle stick risk. However, according to the present invention, once the catheter assembly is in place, a person can push inner tube 19 forwardly in a one-handed operation by grasping the wings 12 with the person's fingers and using the person's palm to push against end 23 of inner tube 19. This causes rib 24 to push past the small locating beads 24 and along passageway 22 (see FIG. 8b).

This forward movement (only a few millimetres) also causes the forward end of inner tube 19 to move towards the stepped portion 13 of outer tube 11 (this being best illustrated between FIG. 8a and FIG. 9a). This forward movement causes the arrowhead configuration on the end of fingers 32 to contact an internal tapering face 34 (see FIG. 9a) which causes the fingers 32 to be pushed inwardly. As this occurs, fingers 32 are released from the inner edge of inner tube 19. As it is these fingers 32 that prevent end member 30 from being sucked back, as soon as the fingers are released, the end member will be sucked back into the distal portion of inner tube 19.

Importantly, as fingers 32 are pushed inwardly and become released from the edge of inner tube 19, they also engage behind the arrowhead formation of fingers 27 which form part of needle holder 26. Thus, as the fingers 32 are pushed inwardly, they lock against fingers 27 and this means that the end member 30 becomes locked to needle holder 26.

Therefore, as end member 30 is sucked back into the distal end of inner tube 19, it also drags back needle holder 26 and therefore the contaminated puncture needle 18, this position being illustrated in FIG. 10 and FIG. 11.

The arrangement of having the needle holder 26 initially attached to outer tube 11 means that the puncture needle 18 exhibits no forward movement by virtue of any of the shoot back mechanism which forms part of the present invention.

FIGS. 12-15 illustrate the turn valve on the catheter. The valve can turn manually between an open position and a closed position as illustrated in the figures.

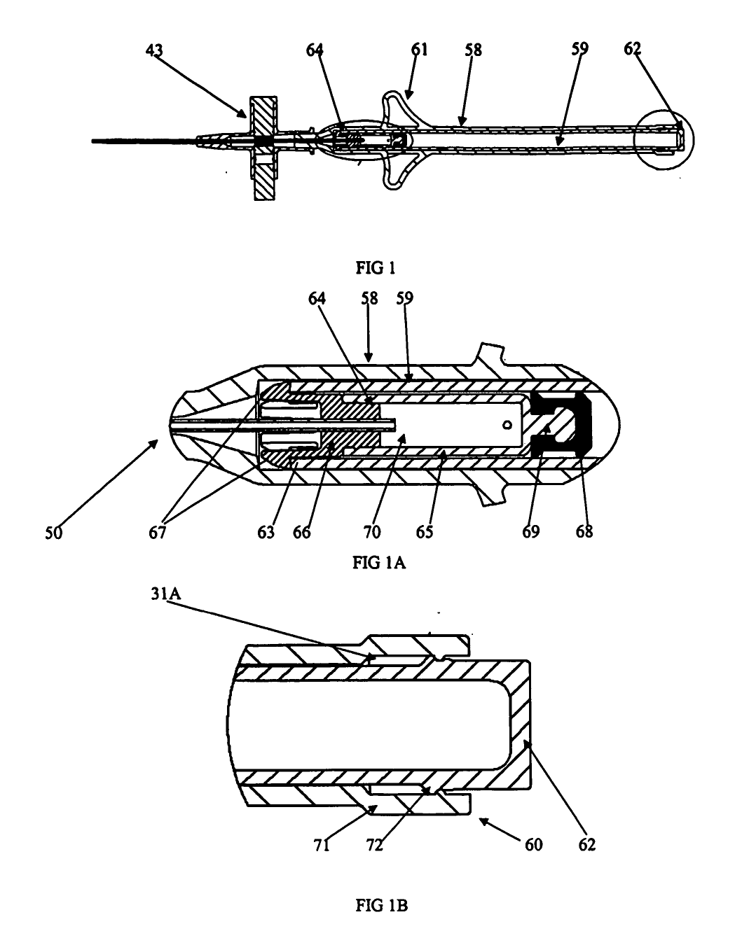

FIGS. 1-7 illustrate a second embodiment of the invention. This embodiment also uses vacuum to suck back the contaminated needle and is similar to the embodiment described with reference to FIGS. 8-16. However, one difference is that instead of a two-part needle holder/end member arrangement which is described in the first embodiment, the second embodiment describes a single unit which in the second embodiment is called a needle holding piston assembly. One difference in the use of the second embodiment of the invention is that triggering the shoot back mechanism does result in the puncture needle moving forward by a few millimetres before being shot back into the inner tube. Occasionally, this slight forward movement can create discomfort to a patient.

The second embodiment (FIGS. 1-7) provides a cannula/catheter introducer combination comprising a cannula and catheter assembly generally indicated by arrow 41 (see FIG. 7) and a retractable needle assembly generally indicated by arrow 42 for connection to the cannula/catheter combination.

The cannula/catheter assembly 41 is provided with a valving member generally indicated by arrow 43 for controlling the ingress and egress of fluids from a patient.

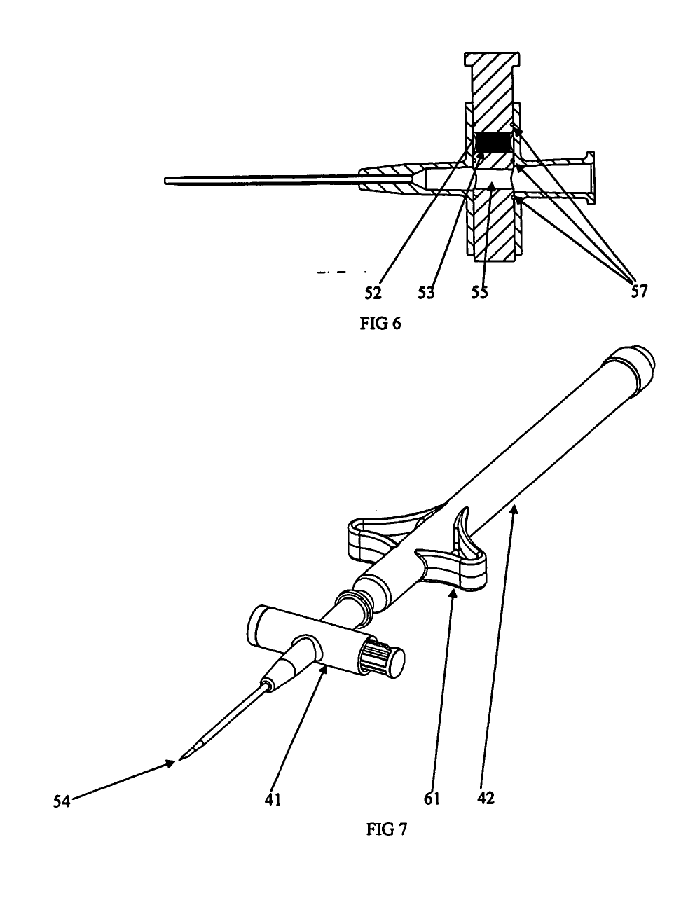

The cannula/catheter assembly is provided with a body 44 having an inlet 45 (FIGS. 5 and 6) and an outlet 46 and the valving member intersects with a valve body 47. The inlet 45 and outlet 46 respectively provide a needle aperture 48 and a slightly tapered socket 49 for connection with an outlet nozzle 50 (see FIG. 1a) of the needle assembly.

The valving member 43 includes a valve plunger 51 which is adjustable from a first sealing position (FIG. 5) and a second flow position (FIG. 6).

The plunger 51 has an aperture 52 therein supporting a resilient pierceable gasket 53. The gasket 53 allows a puncture needle 54 to pass through it and when the needle is withdrawn it closes to seal off the needle penetration.

The plunger is also provided with a peripheral fluted portion 55 which when positioned in the fluid mainstream 16 (see FIG. 6) allows fluid to pass in both directions.

O-ring seals 57 ensure fluid tight sealing.

The retractable needle assembly 42 comprises outer and inner tubes 58, 59 respectively (see FIG. 1) with the inner tube 59 being a sliding fit in the outer tube 58.

The outer tube 58 comprises a bore and has an open rear (distal) end 60 (see FIG. 1b) and is provided with forward finger gripping wings 61. The front (proximal) end of the outer tube has a nozzle 50 that inserts into the socket 49 on the cannula/catheter.

The inner tube 59 has a closed rear (distal) end 62 (see FIG. 1b) and an open front (proximal) end 63 (see FIG. 1a).

A needle holding piston assembly 64 (see FIG. 1a) is a sliding fit within the inner tube 59. The piston assembly 64 comprises a hollow tubular body 65, which has an open front end and a closed rear end. The closed rear end has a mounting knob 69. A sealing member 68 is mounted over the knob 69. A needle mounting block 66 is fitted to the open front end of the body 65.

The piston assembly 64 includes a transparent vial section 70, which enables medical staff to establish that the needle has been correctly inserted when blood flows to the vial and is visually detected.

The open distal end 60 of the outer tube 58 (FIG. 1b) is provided with a shoulder 71 and the closed end 62 of the inner tube 59 has a complementary expanded portion 72 which ensures that the inner tube is secured within the outer tube. Referring especially to FIG. 1b, it can be seen that this arrangement allows the inner tube to be pushed forwardly into the outer tube by a few millimetres, this distance being defined by the length of the recess 31a (which is a few millimetres). The arrangement also ensures that the inner tube 59 cannot be readily pulled out of the outer tube 58. This short stroke of a few millimetres is however sufficient to trigger the shoot back mechanism of the needle as will be described in greater detail below.

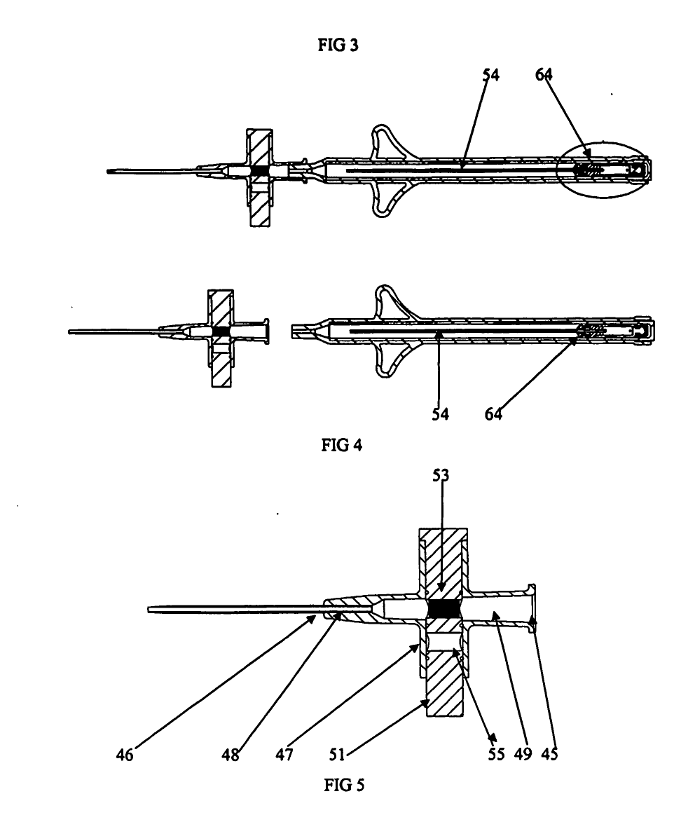

The front end of the needle mounting block 66 is provided with a plurality of raked fingers 67. These fingers are formed integrally with the needle mounting block and are resilient. The fingers are designed to lock against the front of inner tube 59. The function of fingers 67 is to prevent the needle holding piston assembly 64 from moving along the inside of inner tube 59 from the proximal end of the inner tube (see FIG. 1a) to the distal end of inner tube (see, for instance, FIG. 3 and FIG. 4).

A vacuum is provided in inner tube 59 and seal 68 maintains the vacuum inside inner tube 59. Thus, there is a force acting on the needle holding piston assembly 64 to “suck” the needle holding piston assembly 64 back into inner tube 59 towards the distal end of the inner tube; however the resilient fingers 67 lock against the front of inner tube 59 and prevent the needle holding piston assembly 64 from being sucked back.

The arrangement described immediately above and illustrated in FIG. 1 and FIG. 1a, is the initial position of the device.

To dislocate the fingers 67, the end 62 of the inner tube is pushed forward using the palm of the hand whilst gripping the wings 61 with the fingers. The fingers are thereby forced into the tapered section of the nozzle 50 and compressed to the extent that the needle assembly is withdrawn under vacuum, the situation illustrated by FIGS. 3 and 4.

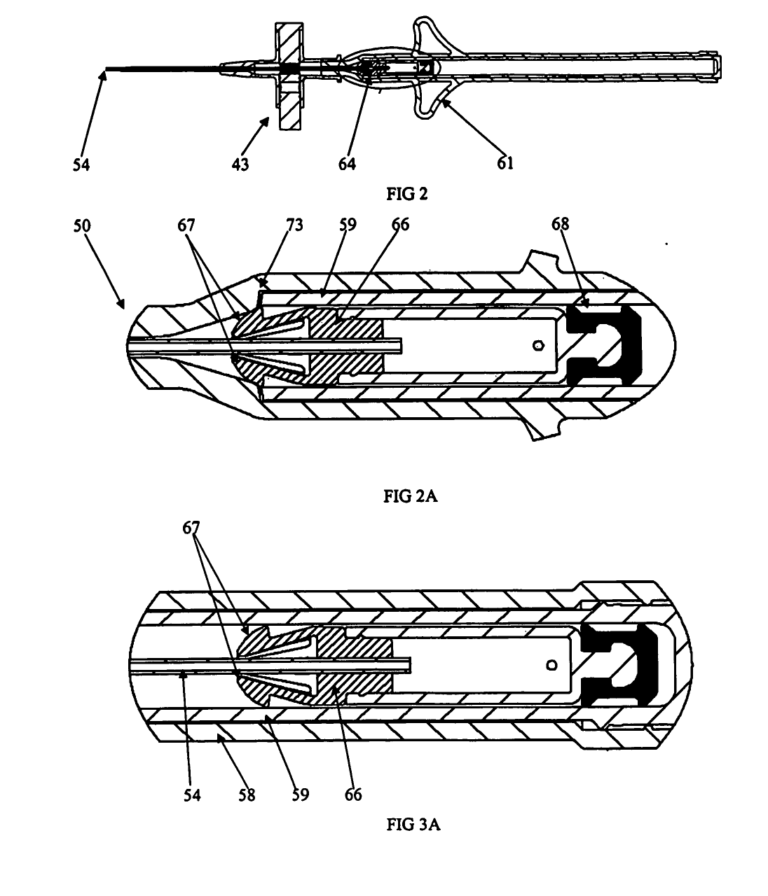

More specifically, inner tube 59 can be pushed forwardly by a few millimetres in the manner described immediately above which moves the expanded portion 72 (see FIG. 1b) from the position illustrated in FIG. 1b, to a position (not illustrated) where the expanded portion is now at the other end of the recess 31a. This short distance is sufficient to trigger the needle shoot back mechanism. Specifically, and best illustrated in FIG. 2a, as the inner tube 59 is pushed forwardly by a few millimetres, it will push the needle holding piston assembly 64 forwardly by a few millimetres. As this occurs, the resilient fingers 67 push forwardly (as they form part of the needle block 66 which is attached to the front of the needle holding piston assembly 64). The front part of outer tube 58 tapers inwardly (see FIG. 1a) and the resilient fingers 67 are arrow shaped in configuration which means that as the resilient fingers are pushed forwardly, they will be pressed inwardly (see FIG. 2a), and consequently they will dislodge from the front of inner tube 59. As soon as the fingers 67 have been released from inner tube 59, the vacuum inside inner tube 59 will suck back the needle holding piston assembly 64 thereby retracting the contaminated puncture needle 54 back into the body of inner tube 59, this position being illustrated in FIG. 3 and FIG. 4.

The forward movement of inner tube 59 is limited by the arrangement illustrated in FIG. 1b, and also by a shoulder 73 (see FIG. 2a) on outer tube 58.

Once the cannula/catheter device is secured and the needle assembly removed, the cannula/catheter can be used for the introduction or withdrawal of fluids to and from a patient.

The arrangement is simple to use and, in practice, once the device has been inserted into the cannula assembly 41, the inner tube 59 is pushed forwardly by just a few millimetres without any requirement to rotate the inner tube or use any separate locking mechanisms and upon forward pushing of the inner tube, the contaminated needle 54 is sucked back to the rear of inner tube 59 to render the contaminated needle 54 safe. It is not possible to push or otherwise enable the contaminated needle 54 to emerge from the safety of inner tube 59. The arrangement is a simple one-handed reliable arrangement.

FIGS. 1 and 2 of the drawings show the fully assembled device as delivered for use, FIG. 3 of the drawings with the needle withdrawn after use, and FIG. 4 separation of the cannula/catheter from the needle assembly.

FIGS. 5 and 6 of the drawings show alternative positions of the plunger 51 of the valving member 43.

In FIG. 5, the plunger 51 is in a first position with the gasket 53 blocking fluid flow.

In FIG. 6, the plunger 51 has been manually repositioned and the fluted portion 55 provides a passage for fluid flow.

FIG. 7 of the drawings is an assembly drawing of the device of the present invention in a ready-to-use condition.

Aspects of the present invention have been described by way of example only and modifications and additions thereto may be made without departing from the spirit or scope thereof.

1. A cannula/catheter introducer comprising
an outer tubular member which has a proximal end and a distal end,
an inner tubular member adapted for sliding movement within the outer tubular member, the inner tubular member having an open proximal end, and a closed distal end,
an end member which closes the open proximal end of the inner tubular member,
a sealing means on the end member to sealingly engage with the inner tubular member,
release means on the end member and which is movable between a first position where the release means locks the end member to the inner tubular member, and a second position where the release means is unlocked from the inner tubular member and allows the end member to retract through the inner tubular member, the inner tubular member being under vacuum, the vacuum functioning to retract the end member from the proximal end of the inner tubular member towards the distal end of the inner tubular member, the release means being movable from the first position to the second position when the inner tubular member is at or adjacent the proximal end of the outer tubular member,
a needle holder which is attached to the proximal end of the outer tubular member, release means on the needle holder and which is moveable between a first position where the release means locks the needle holder to the outer tubular member, and a second position where the release means is unlocked from the inner tubular member,
wherein, when the inner tubular member is at or adjacent the proximal end of the outer tubular member, the release means on the end member unlocks from the inner tubular member, and locks against the release means on the needle holder, and causes the release means on the needle holder to move to the unlocked position, after which the end member and the needle holder is retracted into the inner tubular member by virtue of the vacuum in the inner tubular member.

2. The introducer as claimed in claim 1 wherein the inner tubular member is movable between a retracted position and an extended position, and when in the retracted position, the tubular member is spaced from the needle holder by a short distance, and when in the extended position, the tubular member triggers retraction of the needle holder, the inner tubular member being held captive within the outer tubular member in the retracted position.

3. The introducer as claimed in claim 2 wherein the distal end of the outer tubular member and the distal end of the inner tubular member comprise captive means, the captive means comprising a longitudinal passageway in the outer tubular member and a projection extending from the inner tubular member, the projection adapted for movement along the longitudinal passageway to design the travelling limit of the inner tubular member within the outer tubular member.

4. The introducer as claimed in claim 2 wherein the proximal end of the outer tubular portion is restricted in diameter with respect to the remainder of the outer tubular portion and contains a number of stepped portions to progressively reduce the diameter of the proximal end while still providing internal land portions.

5. The introducer as claimed in claim 2 containing gripping means or locking means on an inner wall at the proximal end of the outer tubular member which function to assist in locking the needle holder to the proximal end of the outer tubular member.

6. The introducer as claimed in claim 5 wherein the gripping means or locking means comprises an internal annular groove in the proximal end of the outer tubular member.

7. The introducer as claimed in claim 2 wherein the end member comprises a rear portion and a front portion, the rear portion being substantially closed and containing the sealing means to enable the end member to be sealing engaged to the inner wall of the inner tubular member in a sliding but sealing manner.

8. The introducer as claimed in claim 7 wherein the front portion contains the release means.

9. The introducer as claimed in claim 8 wherein the release means comprises at least one finger member which is resilient and which is adapted for movement between a natural first position and a deformed second position.

10. The introducer as claimed in claim 9 wherein the at least one finger member, when in the first position, locks against the open proximal wall of the inner tubular member.

11. The introducer as claimed in claim 10 wherein the at least one finger member is provided with an arrowhead configuration to enable the finger member to lock against the open proximal wall of the inner tubular member.

12. The introducer as claimed in claim 1 wherein the needle holder has a central body portion which contains a passageway to accommodate a puncture needle, the release means being formed integrally with the central body portion.

13. The introducer as claimed in claim 12 wherein the release means comprises at least one finger member, the finger member comprising a configuration to enable the finger member to engage with the release means on the end member.

14. The introducer as claimed in claim 13 wherein the finger member contains an intermediate projection that engages with a recess in the outer tubular member to releasably lock the needle holder to the outer tubular member.

15. A cannula/catheter inserter, the inserter comprising
an outer tubular member which has a proximal end and a distal end,
an inner tubular member adapted for sliding movement within the outer tubular member, the inner tubular member having an open proximal end and a closed distal end,
a needle holding piston assembly which closes the open proximal end of the inner tubular member,
a sealing means on the needle holding piston assembly to sealingly engage with the inner tubular member,
release means on the needle holding piston assembly and which is movable between a first position where the release means locks the needle holding piston assembly to the inner tubular member, and a second position where the release means is unlocked from the inner tubular member and allows the needle holding piston assembly to retract through the inner tubular member, the inner tubular member being under vacuum, the vacuum functioning to retract the needle holding piston assembly from the proximal end of the inner tubular member towards the distal end of the inner tubular member, the release means being movable from the first position to the second position upon movement of the inner tubular member towards the proximal end of the outer tubular member,
wherein, upon movement of the inner tubular member towards the proximal end of the outer tubular member, the release means on the needle holding piston assembly unlocks from the inner tubular member, after which the needle holding piston assembly is retracted into the inner tubular member by virtue of the vacuum in the inner tubular member.

16. The inserter as claimed in claim 15 wherein the inner tubular member is movable between a retracted position and an extended position, and when in the retracted position, the tubular member is spaced from the needle holding piston assembly by a short distance, and when in the extended position, the tubular member triggers retraction of the needle holding piston assembly, the inner tubular member being held captive within the outer tubular member such that the inner tubular member can only move by the short distance.

17. A cannula/catheter introducer, the introducer comprising an outer tubular member, an inner tubular member, needle retraction means to enable the puncture needle to be retracted into the inner tubular member upon forward movement of the inner tubular member relative to the outer tubular member, the inner tubular member being substantially inserted into the outer tubular member such that the proximal end of the inner tubular member is adjacent the proximal end of the outer tubular member, the introducer comprising limit means to limit the travel of the inner tubular member relative to the outer tubular member, the limit means being adjacent a distal end of the introducer.

18. The introducer as claimed in claim 1, wherein the release means moves from the first position to the second position by movement of the upper tubular member towards the proximal end of the outer tubular member.
